(19) United States
(12) Patent Application Publication
(10) Pub. No.: US 2010/0175140 A1
(43) Pub. Date: Jul. 8, 2010

(54) Leucine-rich repeat kinase (LRRK2) drosophila model for parkinson's disease: wildtype1 (WT1) and G2019S mutant flies

(75) Inventor: Wanli W. Smith, Bel Air, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/653,986

(22) Filed: Dec. 21, 2009

Related U.S. Application Data
(60) Provisional application No. 61/139,057, filed on Dec. 19, 2008.

(51) Int. Cl. A61K 49/00; A01K 67/00

(52) U.S. Cl. 800/3; 800/13

(57) ABSTRACT

Mutations in the leucine-rich repeat kinase (LRRK2) gene cause late-onset autosomal dominant Parkinson's disease (PD) with pleiomorphic pathology. Previously, we and others found that expression of mutant LRRK2 causes neuronal degeneration in cell culture. Here we used the GAL4/UAS system to generate transgenic Drosophila expressing either wild-type (WT1) human LRRK2 or LRRK2-G2019S, the most common mutation associated with PD. Expression of either WT1 human LRRK2 or LRRK2-G2019S in the photoreceptor cells caused retinal degeneration. Expression of WT1 LRRK2 or LRRK2-G2019S in neurons produced adult-onset selective loss of dopaminergic neurons, locomotor dysfunction, and early mortality. Expression of mutant G2019S-LRRK2 caused a more severe parkinsonism-like phenotype than expression of equivalent levels of WT1 LRRK2. Treatment with L-DOPA improved mutant LRRK2-induced locomotor impairment but did not prevent the loss of tyrosine hydroxylase (TH)-positive neurons. To our knowledge, this is the first in vivo “gain-of-function” model which recapitulates several key features of LRRK2-linked human parkinsonism. These flies may provide a useful model for studying LRRK2-linked pathogenesis and for future therapeutic screens for PD intervention.

This application claims priority benefit of U.S. Provisional Patent application No. 61/139,057 filed Dec. 19, 2008, which is incorporated by referenced herein in its entirety.

This invention was made with government support under Grant No. 1R21NS055684 awarded by the National Institute of health (NIH)/National Institute of Neurological Disorders and Stroke (NINDS). The government has certain rights in the invention.

This invention is illustrated by the following exemplar embodiments, which are not to be construed as imposing limitations on the scope thereof. On the contrary, various other embodiments, modifications, and equivalents thereof, which after reading the description herein, may suggest themselves to those skilled in the art, may be made without departing from the spirit or scope of the present invention.

All publications, patents and patent applications disclosed herein are incorporated into this application by reference in their entirety.

### FIELD OF INVENTION

This invention relates to transgenic Drosophila expressing human Leucine-Rich Repeat Kinase 2 (LRRK2) genes. This is the first in vivo “gain-of-function” model to mimic the LRRK2-linked human PD, which provides the useful model for preclinical compound screens for PD intervention. These screens include identifying the inhibitors of LRRK2 enzyme activities and discovering the neural protective compounds to treat Parkinson's disease

### BACKGROUND OF THE INVENTION

The present invention relates to a transgenic Drosophila expressing a mutation in the leucine rich repeat kinase 2 (LRRK2) protein. The mutant flies recapitulate several key features of Parkinson's disease (PD) such as loss of selective neurons, locomotor dysfunction and early death. Such an animal model may be useful in screening for and testing of substances and pharmaceutical agents that modulate LRRK2 and prevent the progressive death of dopaminergic (DA) neurons in PD.

Mutations in the LRRK2 gene represent the most common known cause of PD (1, 2, 24). Unlike mutations in other PD-linked genes, LRRK2-linked disease has a clinical progression and neurochemical phenotype similar to that of typical late-onset disease, but little is known about LRRK2-linked molecular pathogenesis. The LRRK2 gene spans a 144-kb genomic region, with 51 exons encoding 2527 amino acids. The gene is expressed in all tissues examined, although at low levels. LRRK2 contains multiple conserved domains including MAP-kinase-kinase-kinase (MAPKKK), leucine-rich repeat (LRR), GTPase (ROC and COR), and WD40 domain (1, 2). The discovery of PD-linked point mutations in almost all of the predicted domains of LRRK2, the absence of deletions or truncations, and the dominant inheritance of the disease suggest a ‘gain-of-function’ mechanism for LRRK2-linked PD.

The LRRK2 MAPKKK domain contains sequence homology to both serine/threonine and tyrosine kinases. Several pathogenic mutations of LRRK2 in PD have been found within the protein kinase domain active segment (e.g., G2019S and X2020X), suggesting that these mutations may cause pathology through altering the enzymatic activity of LRRK2 (1, 2, 6). G2019S is the most common mutation in LRRK2 associated PD (7-9) and is believed to increase LRRK2 kinase activity in assays to measure autophosphorylation or phosphorylation of generic substrates (5, 10-14). Abolishing LRRK2 kinase activity diminishes the toxicity of all PD mutants tested in cell culture (11, 12), suggesting that LRRK2 protein kinase activity may play an important role in PD pathogenesis (6).

Increasing evidence suggests that Drosophila melanogaster is an excellent model organism for studying neuronal degenerative diseases (16, 17) such as PD, Alzheimer's disease (AD), tauopathies, amyotrophic lateral sclerosis (ALS), hereditary spastic paraplegia and polyglutamine (polyQ) diseases, and spinocerebellar ataxia (SCA) (17, 18). The LRRK2 gene is highly conserved across species. Drosophila has a single orthologue of the human LRRK2 (CG5483) and a loss-of-function in this gene has been described (19). However, the loss-of-function mutation of Drosophila CG5483 does not constitute an adequate model for the most common forms of LRRK2-linked PD which appear to be “gain-of-function” mutations (like G2019S).

Therefore, there is a need for models that recapitulate the most common form of LRRK2-linked PD in order to facilitate the discovery and evaluation of therapeutic compositions and for testing neuroprotective strategies to prevent neuronal loss and to rescue locomotor dysfunction in PD.

### SUMMARY OF THE INVENTION

The present invention relates to transgenic animals and methods of using transgenic animals. In some embodiments, the transgenic animals are invertebrate transgenic animals, particularly members of the phylum arthropoda, and more particularly members of the class insecta. In preferred embodiments, the models are transgenic flies. In many preferred embodiments, the transgenic flies are members of the family Drosophilidae (e.g., Drosophila melanogaster). The subject invention is further described in terms of transgenic flies employed at any stage of their life, e.g. in the egg stage, in the larval stage, in the adult stage, etc.

In preferred embodiments, the present invention relates to transgenic animal models for neuronal function, in particular to loss of neuronal function, in particular, to a loss of locomotor function. This loss of locomotor function is observed in adult transgenic flies described in the examples herein. The invention provides for transgenic animals that over-express the human WT1 LRRK2 or a LRRK2 variant.

The present invention also provides a method for screening a compound library for compounds that modulate Parkinsonism-like symptoms. This method comprises providing: a) an animal model; and b) one or more test compounds; combining in any order, the animal model and one or more test compounds under conditions such that the animal model and the test compound interact; and detecting alterations in Parkinsonism-like symptoms. In some embodiments, test compounds identified as modulating (e.g., improving) a symptom associated with PD are administered and/or tested in other mammals (e.g., humans) to determine the presence of or elicit a biological response. In some embodiments, the test compounds or sold and/or advertised for use as modulators or potential modulators of the symptom (e.g., for research or therapeutic use).

Compounds or expression of nucleic acids that modify Parkinsonism-like symptoms in Drosophila are also expected to affect PD-related symptoms in humans. A therapeutic method for treating PD may comprise the expression of a nucleic acid, its human homolog(s), or their encoded proteins. In another embodiment, a therapeutic method for treating PD may comprise a substantially purified protein, either recombinant or from a naturally occurring source, encoded by a nucleic acid or its human homolog(s). In yet another embodiment, compounds that affect the activity of the proteins encoded by a nucleic acid or its human homolog(s) may also comprise a therapeutic method for treating PD. In yet another embodiment, antibodies that bind to and affect the activity of the proteins encoded by a nucleic acid or its human homolog(s) may also comprise a therapeutic method for treating PD.

To facilitate understanding of the invention, a number of terms are defined below.

As used herein, the term “animal” refers to any animal (e.g., a mammal), including, but not limited to, humans, non-human primates, rodents (e.g., mice, rats, etc.), insects, (e.g. flies), and the like.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment, are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences”. The mRNA functions during translation to specify the sequence or order of amino acids in a nascent polypeptide.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified”, “mutant”, and “variant” refer to a gene or gene product that displays modifications in sequence and or functional properties (i.e., altered characteristics) when compared to the wild-type gene or gene product. It is noted that naturally occurring mutants can be isolated; these are identified by the fact that they have altered characteristics when compared to the wild-type gene or gene product.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

As used herein the term “coding region” when used in reference to structural gene refers to the nucleotide sequences that encode the amino acids found in the nascent polypeptide as a result of translation of a mRNA molecule. Coding regions in eukaryotes are a composition comprising of 5′ ends with nucleotide triplets “ATG” that encode methionine and 3′ end sequences comprising of nucleotide triplets that specify stop codons (e.g., TAA, TAG, TGA).

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring

The term “transgene” as used herein refers to a foreign, heterologous, or autologous gene that is placed into an organism by introducing the gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “vector” is used in reference to nucleic acid molecules that transfer DNA segment(s) from one cell to another. Vector can include partial genes, gene fragments, full length genes and target sequences. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells utilize promoters, enhancers, and termination and polyadenylation signals.

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “overexpression” and “overexpressing” and grammatical equivalents, are used in reference to levels of mRNA to indicate a level of expression higher than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed, often referred to as “housekeeping” genes (e.g., the amount of 28S rRNA, an abundant RNA transcript present at essentially the same amount in all tissues, present in each sample can be used as a means of normalizing or standardizing the mRNA-specific signal observed on Northern blots.

The term “transfection” as used herein refers to the introduction of foreign DNA into cells. Transfection may be accomplished by a variety of means known to the art including calcium phosphate-DNA co-precipitation, DEAEdextran-mediated transfection, polybrene-mediated transfection, electroporation, microinjection, liposome fusion, lipofection, protoplast fusion, retroviral infection, and biolistics.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise cell lysate, a cell, a portion of a tissue, tissue lysate, whole flies, whole fly extract containing one or more proteins and the like.

### BRIEF DESCRIPTION OF THE DRAWINGS

FIG. 1. LRRK2 induced retinal degeneration. (A) Expression of Flag-LRRK2 and Flag-LRRK-G2019S in photoreceptor cells. Fly head extracts prepared from the indicated transgenic files were fractionated and subjected to Western blot analysis using anti-Flag antibodies. (B) Time course of photoreceptor degeneration determined by the optical neutralization technique. Each data point was based on examination of ≧90 ommatidia from at least six flies. Statistically significant differences between control and LRRK2 or LRRK2-G2019S transgenic flies were indicated: *, P<0.05 by ANOVA. (C) Ommatidia of 5-week-old flies examined by transmission electron microscopy.

FIG. 2. Expression of LRRK2 protein by ddc-GAL4 driver caused locomotor dysfunction. (A) Expression of LRRK2 proteins in flies containing the ddc-GAL4 in combination with the UAS-LRRK2 transgenes. Head extracts from the indicated fly stocks were subjected to Western blot analysis using anti-Flag antibodies [20 μg protein per lane, except for the ddc-GAL4;LRRK2-4 (10 μg)]. (B). Survival curves of files expressing either LRRK2 or LRRK2-G2019S (n=50). (C). Cohorts of 60 flies from each genotype were subjected to climbing assays weekly. Statistically significant differences between the control and LRRK2 transgenic lines (except the 4 week data) are indicated: *, P<0.05 by ANOVA. Statistically significant differences between LRRK2-1 and G2019S-2 flies are indicated: #, P<0.05 by ANOVA. (D). Cohorts of 20 flies from each genotype at five week of age were subjected to the actometer to measure locomotor activity. Shown are representative data from three separate experiments. (E). Flies at 5 week of age were untreated or treated with 1 mM L-DOPA for 10 days, then subjected to climbing assays. Statistically significant differences between untreated ddc-GAL4;G2019S-2 flies were indicated: *, P<0.05 by ANOVA.

FIG. 3. Expression of LRRK2 protein by ddc-GAL4 driver induced loss of TH positive dopaminergic neurons. (A) Diagram of DA and 5-HT neuron clusters in the medial and lateral areas of the adult fly brain as in the previous publications (35, 36). (Left) Five clusters: PPM1 (unpaired), PPM2 (paired), PPM3 (paired) (protocerebralposterior medial); PPL1 and PPL2 (paired; protocerebral posterolateral) on the posterior side. (Center) two DA clusters: PAL (protocerebral anterolateral) and PAM (paired anterolateral medial) on the anterior side. (Right) Five distinct 5-HT neuronal clusters (SP1, SP2, LP1, LP2 and IP) in the two brain hemispheres. (B-D), Dissected whole brains were subjected to anti-TH immunofluorescent staining. (B). Quantitation of TH-positive neurons in PPM 1/2 clusters in transgenic flies of the indicated ages. (C). Average numbers of TH-positive neurons per DA cluster in 5 week old flies of the indicated genotypes. (D). Representative images of anti-TH staining in PPM1 and PPM2 clusters from 5 week old flies of the indicated genotypes. Statistically significant differences between the control and all LRRK2 transgenic lines were indicated: *, P<0.05 by ANOVA. Statistically significant differences between LRRK2-1 and G2019S-2 flies were indicated: #, P<0.05 by ANOVA.

FIG. 4. Expression of LRRK2 by elav-GAL4 driver caused late-onset locomotor impairment. (A and B), Expression of CG5483 and human LRRK2 in various types of fly brain tissues. Total RNA was prepared from fly brain tissues and cDNA was generated. Semi-quantitative RT-PCR was performed using primers for CG5483 and human LRRK2 to assess mRNA levels. (A), Representative image of RT-PCR products. (B) Quantitative analysis of relative mRNA levels of CG5483 and human LRRK2. *, P<0.05 vs UAS-LRRK2 by ANOVA. (C) LRRK2 autophosphorylation (kinase) analysis of various fly head homogenates. Anti-Flag-LRRK2 immunoprecipitated samples from fly head homogenates were incubated with [γ-³²P]ATP, subjected to SDS-polyacrylamide gel electrophoresis (SDS-PAGE) and blotted onto polyvinylidene difluoride (PVDF) membranes. The samples were then imaged using a phosphoimaging system. The incorporation of [y-³²P]ATP into LRRK2 protein increased by −2.8-fold in elav-GAL4;G2019S-2 flies compared with elav-GAL4;LRRK2-1 flies. Shown are representative images from three independent experiments. (D). Cohorts of 60 flies from each genotype were subjected to climbing assays weekly. Statistically significant differences between the control and all LRRK2 transgenic lines are indicated: *, P<0.05 by ANOVA. Statistically significant differences between LRRK2-1 and G2019S-2 flies are indicated: #, P<0.05 by ANOVA. (E). Survival curves of flies expressing either LRRK2 or LRRK2-G2019S (n=50).

FIG. 5. Expression of LRRK2 in all neurons caused selective loss of anti-TH positive neurons. (A) Anti-elav whole-mount brain immunofluorescence of flies at 5 weeks after eclosion showed that expression of either LRRK2-1 or G2019S-2 did not significantly change the density of immunofluorescence. There was a slight but no significant decrease in anti-elav staining in LRRK2 transgenic flies. This could be due to loss of anti-TH positive neurons. (B) Anti-5-HT whole mount brain immunofluorescence of flies at 5 week after eclosion showed that expression of either LRRK2-1 or G2019S-2 did not cause loss of 5-HT positive neurons. (C) Anti-TH whole-mount brain immunofluorescence of flies at 5 week post eclosion showed that expression of G2019S induced loss of TH-positive neurons. (D) Shown are representative images of whole-mount brain sections of flies 5 week post eclosion. (Left) Anti-elav staining brain section. (Center): SP1, SP2 and IP 5-HT neuronal clusters with anti-5-HT staining. (Right): PPM1 and PPM2 TH-stained DA neuronal clusters.

FIG. 6. Sequence Listing. FIG. 6 lists sequences identified herein.

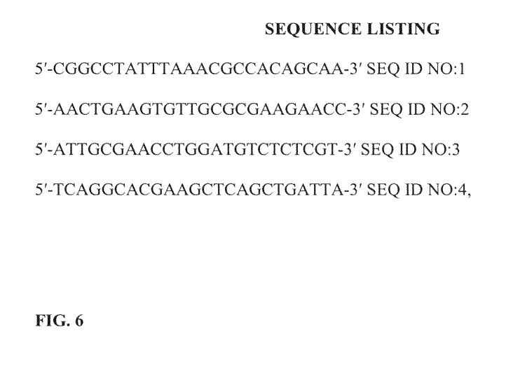

### DETAILED DESCRIPTION OF THE INVENTION

The present invention provides transgenic animals that exhibit a Parkinsonism-like phenotype, having somatic and/or germ cells in which human LRRK2 genes are expressed. The present invention also provides drug-screening assays.

The present invention relates to transgenic animals and methods of using transgenic animals. In some embodiments, the transgenic animals are invertebrate transgenic animals, particularly members of the phylum arthropoda, and more particularly members of the class insecta. In preferred embodiments, the models are transgenic flies. In many preferred embodiments, the transgenic flies are members of the family Drosophilidae (e.g., Drosophila melanogaster). The subject invention is further described in terms of transgenic flies employed at any stage of their life, e.g. in the egg stage, in the larval stage, in the adult stage, etc.

In recent years, the usability of a fruit fly, Drosophila melanogaster, has been much publicized in comparison with that of mammalian models such as mice. Originally, the use of Drosophila was established as an experimental system for transformation by genetic breeding in the field of genetics in a manner similar to those for corn and peas. Recently, it has begun to be shown that a mutation in an individual trait of Drosophila can reflect a human trait as well or better than a mammalian model, depending on the characteristics of the relevant gene group. In particular, Drosophila is highly useful for gene groups involved in the above-described signal transduction, signal transduction system and cellular life span.

Neurodegenerative diseases or disorders according to the present invention comprise Alzheimer's disease (AD), Parkinson's disease (PD), Huntington's disease, amyotrophic lateral sclerosis, Pick's disease, fronto-temporal dementia, progressive nuclear palsy, corticobasal degeneration, cerebro-vascular dementia, multiple system atrophy, and mild-cognitive impairment. Further conditions involving neurodegenerative processes are, for instance, ischemic stroke, age-related macular degeneration, and narcolepsy.

A preferred embodiment of the present invention is based upon a non-mammalian animal model for PD in the fruit fly Drosophila melanogaster (hereafter referred to as Drosophila). Many genes implicated in human diseases, including signaling pathways and effectors of tissue- and cell-specification, were originally identified and characterized in the fruit fly. Thus, genes within most human disease-associated networks are present in the fruit fly genome and have comparable roles in fly biology.

The benefits of using a Drosophila animal model system in some preferred embodiments of the present invention have been exploited by the present invention to study PD. This PD model has tremendous utility in identifying PD causing genes, modifier genes of PD-related symptoms and therapeutic compounds.

In one aspect, the present invention features a non-human animal that expresses a human gene coding for WT1 Leucine-rich repeat kinase 2 (LRRK2) or a G2019S (GS2) mutation. In a preferred embodiment, said animal is an invertebrate. Preferably, said animal is an insect, in particular a fly.

The present invention provides transgenic animals that exhibit a parkinsonism-like phenotype , wherein the parkinsonism-like phenotype includes selective dopaminergic (DA) neuron loss, retinal degeneration, locomotor dysfunction and premature mortality.

The present invention is described below:

A transgenic Drosophila strain expressing a WT1 human Leucine-rich repeat kinase 2(LRRK2) having the following properties: expression of WT1 LRRK2 creates a parkinsonism-like phenotype and wherein said fly exhibits selective dopaminergic (DA) neuron loss, retinal degeneration, locomotor dysfunction and premature mortality.

A second transgenic Drosophila strain expressing a human LRRK2 G2019S mutation having the following properties: creates a parkinsonism-like phenotype and said fly exhibits selective DA neuron loss, retinal degeneration, severe locomotor dysfunction and premature mortality.

A third transgenic Drosophila strain expressing a human LRRK2 12020T mutation having the following properties: creates a parkinsonism-like phenotype and said fly exhibits selective DA neuron loss, retinal degeneration, severe locomotor dysfunction and premature mortality.

Another embodiment of this invention encompasses a transgenic Drosophila that is expressing LRRK2 in all neurons under the control of the elav-GAL4. A second embodiment of this invention encompasses a transgenic Drosophila that is expressing LRRK2 in DA neurons under the control of the ddc-GAL4.

In a further preferred embodiment, the expression of said human LRRK2 (WT1 or G2019S) gene results in an identifiable phenotype in said animal. The identifiable phenotype is related to Parkinsonism-like disorders.

In a further embodiment, the expression of said human LRRK2 (WT1 or GS2) gene results in an identifiable phenotype in said animal that can be accelerated through genetic manipulation or environmental stressors (e.g. oxidative stress, insecticides, 1-methyl-4-phenyl-1,2,3,6-tetrahydropyridine, Nitro oxicide (NO) donor, proteasome inhibitors). The identifiable phenotype is related to Parkinsonism-like disorders.

Accordingly, in some embodiments, the present invention provides animals with transgenic somatic and germ cells having a functional disruption of at least one, and more preferably both, alleles of an LRRK2 gene. Accordingly, the invention provides adult viable animals having a mutated, truncated, partially deleted or fully deleted, or disrupted LRRK2 gene, with reduced (e.g., eliminated) LRRK2 protein production and/or activity. The invention provides adult viable animals having a mutated, truncated, partially deleted or fully deleted, or disrupted LRRK2 gene, with increased LRRK2 protein production and/or activity. In some embodiments the LRRK2 gene is a human LRRK2 gene. A preferred embodiment is a fly comprising a human wild type LRRK2 gene. A preferred embodiment is a fly comprising a human LRRK2 gene with at least one point mutation in the protein kinase domain active segment. A more preferred embodiment is a fly comprising a human LRRK2 gene that expresses LRRK2 with a G2019S mutation.

In one embodiment, LRRK2 gene is expressed such that expression of the LRRK2 gene product is substantially increased in cells of the animal. In a preferred embodiment the LRRK2 is exogenous. In a more preferred embodiment the LRRK2 expressed in the fly is a human LRRK2.

In another embodiment, the animal may be heterozygous or, more preferably, homozygous for the LRRK2 gene disruption. As used herein, the term “gene disruption” refers to any genetic alteration that prevents normal production of LRRK2 protein (e.g., prevents expression of a LRRK2 gene product, expression of normal LRRK2 gene product, or prevents expression of normal amounts of the LRRK2 gene product). In some embodiments, the gene disruption comprises a deletion of all or a portion of the LRRK2 gene. In other embodiments, the gene disruption comprises an insertion or other mutation of the LRRK2 gene. In still other embodiments, the gene disruption is a genetic alteration that prevents expression, processing, or translation of the LRRK2 gene. In one embodiment, both LRRK2 gene alleles are functionally disrupted such that expression of the LRRK2 gene product is substantially reduced or absent in cells of the animal. The term “substantially reduced or absent” is intended to mean that essentially undetectable amounts of normal LRRK2 gene product are produced in cells of the animal. This type of mutation is also referred to as a “null mutation” and an animal carrying such a null mutation is also referred to as a “knockout animal.” In preferred embodiments, the transgenic animals display a Parkinson's disease phenotype similar to that observed in humans.

In various contemplated embodiments, the Drosophila melanogaster strain expressing human LRRK2 WT or LRRK2 G2019S can be genetically crossed with other strains of Drosophila, which is able to transmit a trait introduced by the transferred LRRK2 gene to the offspring, and which is able to inherit the inherited traits of other Drosophila strains to be crossed therewith.

Any method for using transgenic animals to generate models and methods for genetic screening and drug screening is contemplated by the present invention.

In some embodiments, phenotypic changes are modified by compositions (e.g., drugs). In some embodiments, the locomotor function is modified by compositions (e.g., drugs).

In yet another embodiment, compounds that affect the activity of the proteins encoded by a nucleic acid or its human homolog(s) may also comprise a therapeutic method for treating PD. In yet another embodiment, antibodies that bind to and affect the activity of the proteins encoded by a nucleic acid or its human homolog(s) may also comprise a therapeutic method for treating PD.

The present invention also provides a method for screening a compound library for compounds that modulate Parkinsonism-like symptoms. This method comprises providing: a) an animal model; and b) one or more test compounds; combining in any order, the animal model and one or more test compounds under conditions such that the animal model and the test compound interact; and detecting Parkinsonism-like symptoms including neuronal cell loss, locomotion disorders and early death. In some embodiments, test compounds identified as modulating (e.g., improving) a symptom associated with PD are administered and/or tested in other mammals (e.g., humans) to determine the presence of or elicit a biological response. In some embodiments, the test compounds or sold and/or advertised for use as modulators or potential modulators of the symptom (e.g., for research or therapeutic use).

The present invention provides methods and compositions for using transgenic animals as a target for screening drugs that can alter, for example, interaction between LRRK2 and binding partners. Drugs or other agents (e.g., from compound libraries) are exposed to the transgenic animal model and changes in phenotypes or biological markers are observed or identified. For example, drugs are tested for the ability to improve neurological function or phenotypes associated with loss of neurological function.

The present invention further provides agents identified by the above-described screening assays. Accordingly, it is within the scope of this invention to further use an agent identified as described herein in an appropriate animal model (e.g., LRRK2 WT and GS2 transgenic animals) to determine the efficacy, toxicity, side effects, or mechanism of action, of treatment with such an agent. Furthermore, agents identified by the above-described screening assays can be used for treatments of neurologically related disease (e.g., including, but not limited to, Parkinson's disease).

### Material and Methods

Generation of Human LRRK2 Transgenic Flies.

To generate UAS-LRRK2 and UAS-LRRK2-G2019S transgenic flies, the genes encoding these human proteins with N-terminal Flag tags were excised from pcDNA3.1 vectors, cloned between the XhoI site of pUAST vector (20), and verified by sequencing. The resulting constructs were microinjected in w¹¹¹⁸ fly embryos (Rainbow Transgenic Flies). We obtained two transgenic lines each of UAS-LRRK2 and UAS-LRRK2-G2019S. The LRRK2 expression levels were examined by anti-Flag Western blot analysis. We used elav-GAL4, ddc-GAL4 (32), and GMR-GAL4 (33) to express UAS-LRRK2 and UAS-LRRK2-G2019S in all neurons, DA neurons, and photoreceptor cells, respectively. We selected two representative WT (LRRK2-1 and LRRK2-4) and mutant (G2019S-2 and G2019S-3) lines to conduct the phenotype characterization. Drosophila were grown on standard cornmeal medium at 25° C.

Western Blot Analysis.

Adult fly heads were homogenized at 4° C. in buffer A (50 mM Tris.HCl, pH 7.5/1 mM EGTA/0.5 M NaCl/1% Triton X-100/1 mM DTT with protease inhibitors) and extracted as described (33). The resulting homogenates were subjected to Bradford protein assays to ensure equal protein loading and resolved on 4-12% SDS/NuPAGE Bis-Tris gels and transferred onto PVDF membranes (Invitrogen). The membranes were blocked in TBST (pH 7.4, 10 mM Tris.HCl/150 mM NaCl/0.1% Tween 20) containing 5% nonfat milk and then probed with anti-Flag antibody (Sigma). Proteins were detected by using enhanced chemiluminescence reagents (NEN).

Optical Neutralization Technique.

Adult fly heads were mounted on microscope slides using clear nail varnish and observed under a light microscope (33). To obtain a semi-quantitative and unbiased index of retinal degeneration, the investigator counting the number of visible rhabdomeres did not know the genotype of the samples. The mean number of rhabdomeres per ommatidium was calculated. Rhabdomeres were counted by using cohorts of six flies of each genotype weekly during the lifespan of flies.

Electron Microscopy.

Fly heads were hemisected under a dim red photographic safety light, fixed (2% paraformaldehyde/2% glutaraldehyde in 0.1 M sodium cacodylate/3 mM calcium chloride, pH 7.2) at 4° C. overnight, and postfixed in reduced osmium tetroxide for 1 h. Samples were stained en bloc with 2% uranyl acetate (filtered) and dehydrated through a graded series of ethanol. Eyes were oriented in gelatin capsules (size 00) and cured at 50° C. for 24 h. Blocks were sectioned on a Riechert Ultracut E with a low-compression Diatome Diamond knife. Eighty-nanometer sections were picked up on copper slot grids and stained with uranyl acetate followed by lead citrate. Grids were viewed on a Hitachi 7600 TEM operating at 80 kV, and digital images were captured with an AMT 1 K×1 K CCD camera as described (33).

Survival Curve.

Cohorts of 50 flies from each genotype were monitored for survival. Flies were maintained on standard media, and fresh food media were changed every 3-4 days. Mortality was scored daily and analyzed by using Kaplan-Meier survival curves. This experiment was repeated once.

Climbing Assay.

We determined locomotor ability using a climbing assay (negative geotaxis assay) as described previously (23). Cohorts of 60 flies from each genotype were subjected to the assay weekly from 1 week to the time of death. The tested flies were age-matched, randomly selected, anesthetized, and placed in a vertical plastic column (length, 25 cm; diameter, 1.5 cm). After a 30-min recovery from CO₂ exposure, flies were gently tapped to the bottom of the column. We counted and calculated the percentage of flies that could climb to or above the median line of the cylinder in 10 seconds. Each week, the assay was repeated three times.

Actometer Test.

Cohorts of 20 flies from each genotype were subjected to the actometer assay at 5 weeks of age. A single fly was placed in a small tube with food at one end and was monitored for 3 days under standard conditions of 12-h light and 12-h darkness intervals. Activity was recorded on the computer every time the fly crossed an infrared beam (locomotion actograms), and the data were grouped into 30-min bins as described (34).

Real-Time RT-PCR.

To determine the expression of CG5483 and human LRRK2 at the mRNA level, primers were designed targeting CG5483 (5′-CGGCCTATTTAAACGCCACAGCAA-3′) SEQ ID NO:1 and (5′-AACTGAAGTGTTGCGCGAAGAACC-3′) SEQ ID NO:2 and human LRRK2 (5′-ATTGCGAACCTGGATGTCTCTCGT-3′) SEQ ID NO:3 and (5′-TCAGGCACGAAGCTCAGCTGATTA-3′) SEQ ID NO:4, respectively. The semiquantitative real-time RT-PCR was performed by using Stratagene Mx3000P PCR motion and Brilliant II QRT-PCR Master Mix kit according to the manufacturer's protocol.

Immunoprecipitation (IP) and in Vitro Autophosphorylation (Kinase) Assays.

IP experiments from fly head homogenates were performed with anti-FLAG-agarose (Sigma). Precipitates were washed twice with lysis buffer and twice with kinase assay buffer (Cell Signaling Technology). A kinase activity assay was described previously using autophosphorylation because the authentic substrate(s) is not yet known (12). Briefly, kinase reactions were carried out for 90 min at 30° C. in 40 μl of kinase assay buffer with the addition of 15 μl of solution containing 50 mM MgCl₂, 500 μM ATP, and 10 μCi of [γ-³²P]ATP (3,000 Ci/mmol). Reactions were stopped by the addition of Laemmli sample buffer and boiling for 5 min. Samples were separated on 4-12% SDS/PAGE and blotted onto PVDF membranes. Quantification was performed with a phosphoimager (Bio-Rad Molecular Imager FX).

Immunostaining and Cell Counting.

Fluorescent immunostaining was performed on whole-mount dissected adult brain (23, 35) at 1, 21, 35, and 49 weeks of age. Cohorts of six to eight flies per genotype were used at each time point for immunostaining. The dissected brains were mounted in Vectashield (Vector Laboratories).

Rabbit polyclonal anti-TH (Chemicon), mouse monoclonal anti-TH (Immunostar), anti-5-HT (Sigma), anti-elav (Developmental Studies Hybridoma Bank), and anti-Flag-antibodies were used as the primary antibodies. Alexa Fluor 488 goat anti-mouse IgG and Alexa Fluor 568 goat anti-mouse IgG (Invitrogen) were used as secondary antibodies. The numbers of DA and 5-HT neurons were scored in whole-mount brains under fluorescent (Zeiss LSM 250) and/or confocal microscopy (Zeiss LSM 510). For quantification of loss of anti-elav staining in brain, entire brain sections were digitized with an image analysis system. NIH Image J software was used to measure the optical density of anti-elav staining within the entire brain section (six brain sections per experimental group).

Data Analysis.

Quantitative data were expressed as arithmetic means±SEM based on at least three separate experiments. Statistically significant differences between two groups were analyzed by ANOVA. A P value<0.05 was considered significant.

### Examples

The following examples are provided in order to demonstrate and further illustrate certain preferred embodiments and aspects of the present invention and are not to be construed as limiting the scope thereof.

A non-mammalian animal model for PD recapitulates many important aspects of human PD-related symptoms.

### Example 1

### LRRK2 Induces Retinal Degeneration

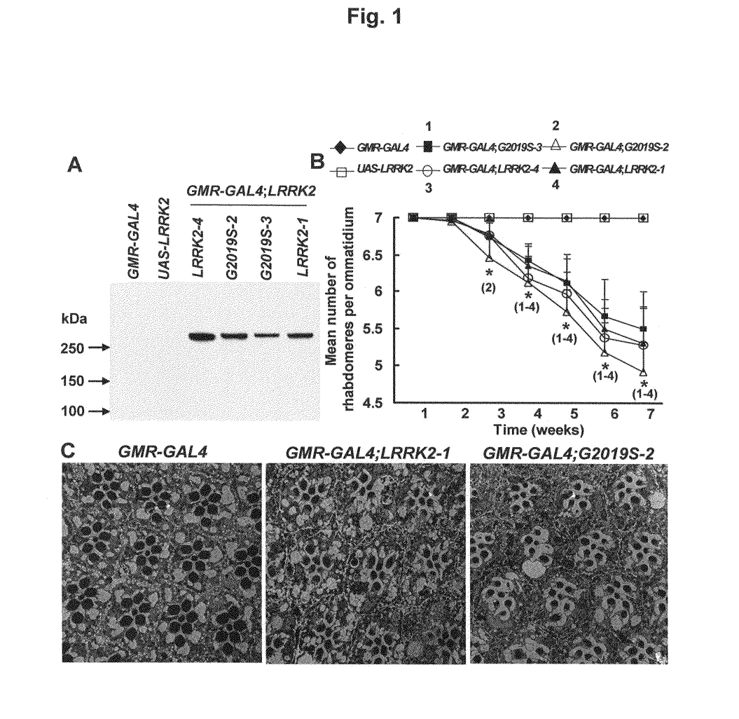

To address whether overexpression of wild-type human LRRK2 and the mutant LRRK2-G2019S phenocopy the human disease in flies, we introduced these proteins in specific subsets of cells using the GAL4/UAS system (20). This system takes advantage of the yeast GAL4 transcription factor, which binds specifically to the upstream activation sequence (UAS). Thus, UAS-linked transgenes can be expressed in specific cell types under the control of a given promoter (promoter-GAL4). To determine whether introduction of LRRK2 and LRRK2-G2019S causes a parkinsonism-like phenotype, we first assayed for retinal degeneration, because photoreceptor cell death has been used to assay neurodegeneration in other fly models of PD (18, 21). Therefore, we expressed two lines of UAS-LRRK2 (1 and 4) and two lines of UAS-LRRK2-G2019S (2 and 3) in photoreceptor cells, under the control of the glass multiple reporter (GMR)-GAL4. Using antibodies directed against the N-terminal Flag tags, we found that the wild-type and mutant proteins were stably expressed (FIG. 1A). The fly compound eye comprises ≈800 repeat units, ommatidia, each including seven photoreceptor cells in any given plane of section. Each photoreceptor cell has a microvillar structure, the rhabdomere, which is the site of photoreception and is the invertebrate equivalent of the rod and cone outer segment. To examine the kinetics of retinal degeneration, we used the optical neutralization technique. As shown, retinal degeneration was detectable by 3 weeks after eclosion in LRRK2 transgenic flies and increased markedly in older flies in comparison with GMR-GAL4 or UAS only control flies (FIGS. 1B and C).

### Example 2

### Expression of LRRK2 by ddc-GAL4 Causes Early Mortality and Locomotion Impairment

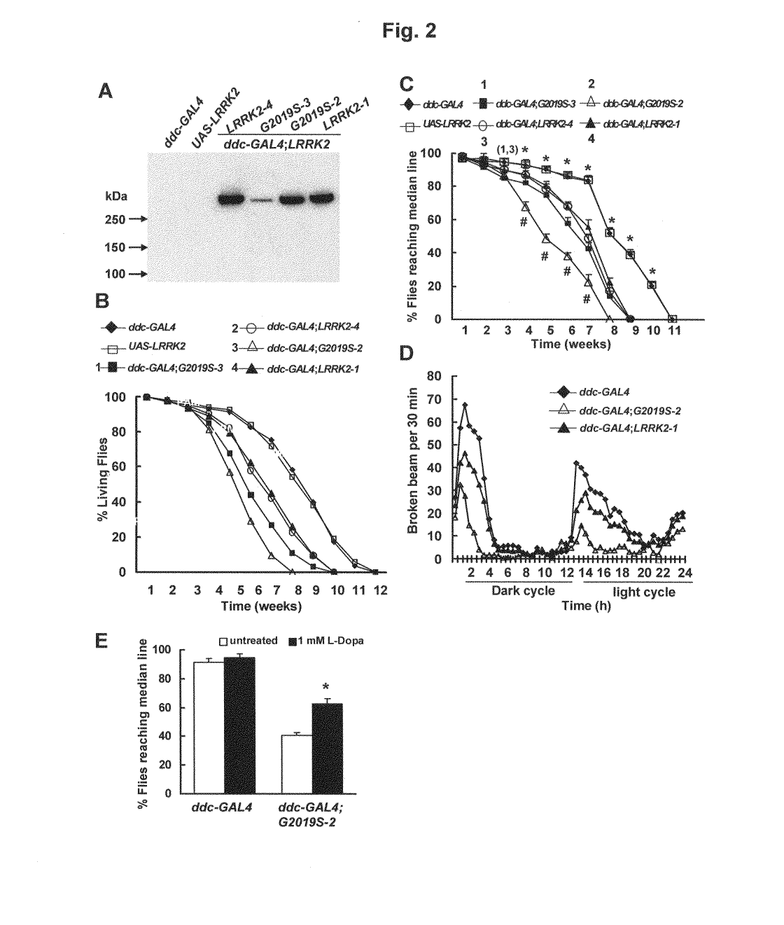

To express LRRK2 in DA neurons, we combined the UAS-WT-LRRK2 and UAS-G2019S-LRRK2 transgenes with the dopa decarboxylase (ddc)-GAL4 driver. Using an anti-Flag antibody, both wild-type and mutant LRRK2 were readily detected in fly head homogenates (FIG. 2A). Survival curves used to examine whether expression of either LRRK2 or LRRK2-G2019S in DA neurons affected fly viability revealed that expression of either LRRK2 or LRRK2-G2019S caused premature mortality (FIG. 2B), although expression of mutant LRRK2-G2019S caused more severe mortality at equivalent expression level (FIGS. 2A and B). The ages at which 50% of the LRRK2-1 and G2019S-2 transgenic flies survived were 48 and 38 days, respectively. The G2019S-3 line had much lower expression than wild-type LRRK2-1 but had a faster rate of mortality (FIG. 2 B). To measure the behavioral differences resulting from expression of LRRK2 in DA neurons, we used a climbing assay (negative geotaxis test). When tapping the flies to the bottom of the vial, nearly all control flies (ddc-GAL4 or the UAS-LRRK2) that were <7 weeks old climbed rapidly to the top of the vial (FIG. 2C). As the control flies aged, they were no longer able to climb to the top of the vial but instead made short abortive climbs and fell back to the bottom of the vial. We found that young (≦7 days old) flies expressing either wild-type or mutant LRRK2-G2019S climbed as well as nontransgenic control flies. However, over time, their performance declined more rapidly than that of the control flies, revealing a locomotor dysfunction of the LRRK2 transgenic flies (FIG. 2C). Moreover, G2019S-2-expressing flies displayed more severe impairment than flies expressing wild-type LRRK2-1 at comparable levels (FIG. 2C).

To further assess the deficits in locomotor activity, we used an actometer to assess the locomotor activity in 5-week-old flies during a 12-h dark/12-h light cycle. The control flies displayed two peaks of activity, and expression of either wild-type or LRRK2-G2019S did not affect the times at which peak activity occurred. By contrast, expression of the LRRK2 transgenes in DA neurons decreased the frequency of locomotor activity (FIG. 2D). Consistent with the climbing assay, the activity of the flies expressing LRRK2-G2019S was more severely impaired than that of flies expressing wild-type LRRK2 at equivalent levels (FIG. 2D). Finally, treatment of ddc-GAL4;G2019S-2 flies (5 weeks of age) with 1 mM L-DOPA for 10 days significantly improved the locomotor activity of ddc-GAL4;G2019S-2 flies (FIG. 2E).

### Example 3

### LRRK2 Induces DA Neuronal Degeneration

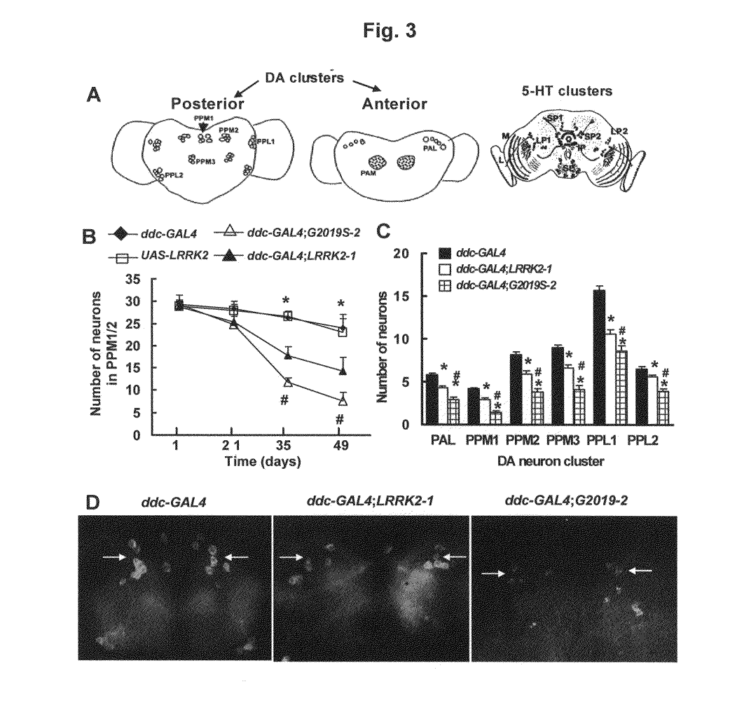

Six neuronal DA clusters are normally present in each Drosophila adult brain hemisphere (22, 23). These neurons express tyrosine hydroxylase (TH), which is an enzyme required for the biosynthesis of dopamine. Flag-LRRK2-linked immunofluorescence was evident in neurons of all DA neuron clusters and colocalized with anti-TH immunostaining (data not shown). To assess whether expression of LRRK2 resulted in degeneration of DA neurons, brains from transgenic flies at 1, 21, 35, and 49 days after eclosion were dissected and immunostained with anti-TH antibodies. In control flies (ddc-GAL4 or UAS-LRRK2 flies), the DA clusters did not change significantly in number or morphology during aging, as monitored by anti-TH staining (FIG. 3B). At 1 day after eclosion, there were no differences in anti-TH-positive staining between the control and LRRK2 or LRRK2-G2019S flies (FIG. 3B). However, at 5 weeks of age, anti-TH staining decreased significantly in flies expressing either wild-type or mutant LRRK2-G2019S (FIG. 3B-D). We counted the TH-positive cells in all clusters except in the paired anterolateral medial (PAM) cluster, because the density of the neurons in PAM was too high to allow precise quantification. We found statistically significant TH-positive neuronal loss in all of the DA clusters examined (FIG. 3C). In addition, mutant LRRK2-G2019S caused more TH-positive neuronal loss than wild-type LRRK2 at equivalent expression levels (FIG. 3B-D). Although L-DOPA improved the mutant LRRK2-induced locomotor impairment, it did not prevent the loss of TH-positive neurons (data not shown). Because ddc-GAL4 can also lead to LRRK2 transgenes expressing in serotonin (5-HT) neurons, we also examined whether LRRK2 protein affected 5-HT neurons using anti-5-HT whole-mount brain immunohistochemical analysis. We found that the brains of flies expressing either wild-type or mutant LRRK2-G2019S at 5 and 7 weeks after eclosion displayed 5-HT immunoreactivity (data not shown) similar to that of the control flies.

### Example 4

### Expression of LRRK2 in All Neurons Causes Late-Onset Locomotion Impairment and Selective Loss of TH-Positive Neurons

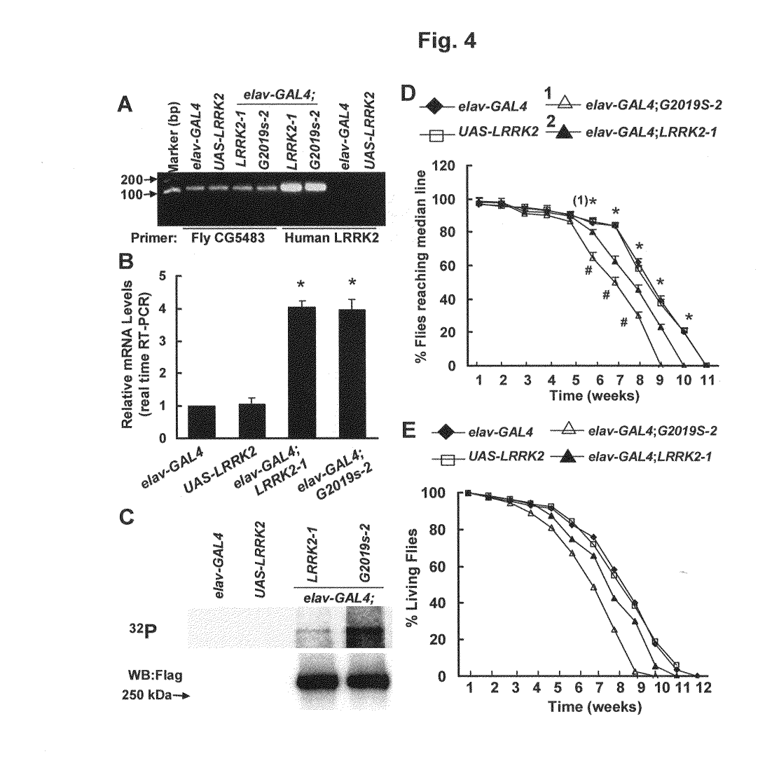

To further determine the effect of LRRK2, we expressed LRRK2 protein using the panneuronal driver, the embryonic lethal abnormal visual system gene (elav)-GAL4, achieving comparable levels of expression in elav-GAL4;LRRK-1 and elav-GAL4;G2019S-2 fly brains by RT-PCR (FIGS. 4A and B) and anti-Flag Western blot (FIG. 4 C). The human LRRK2 transgene transcripts were more abundant than CG5483 (FIGS. 4A and B). Importantly, we found that the protein kinase activity in homogenates from elav-GAL4;G2019S-2 fly heads was ≈2.8-fold higher than that measured in elav-GAL4;LRRK2-1 (FIG. 4C). At 1 week after eclosion, flies expressing either wild-type or mutant LRRK2 under the control of elav-GAL4 displayed normal locomotor activity (FIG. 4D). However, by 6 weeks, the climbing assay showed significant motor impairment in elav-GAL4;G2019S-2 flies and a slight deficit in elav-GAL4;LRRK2-1 flies compared with control flies (FIG. 4D). The climbing performance of flies expressing either LRRK2-1 or LRRK2-G2019S-2 declined more rapidly than that of control flies. Moreover, the mutant LRRK2-G2019S flies exhibited more severe impairment than did the wild-type flies 6 week after eclosion (FIG. 4D). We also found that expression of LRRK2-1 and LRRK2-G2019S-2 shortened the lifespan compared with control flies (FIG. 4 E). The ages at which 50% of the LRRK2-1 and G2019S-2 transgenic flies survived were 49 and 55 days, respectively.

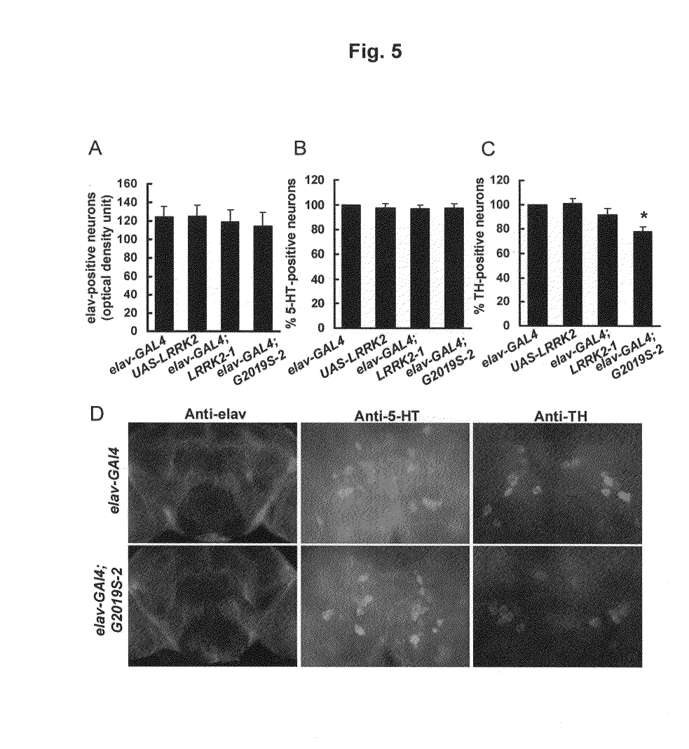

Analysis of brains of elav-GAL4;G2019S-2 flies at 5 weeks of age revealed reductions in TH immunoreactivity up to 22% in DA clusters whereas the elav-GAL4;LRRK2-1 flies showed only a slight decrease (FIGS. 5C and D). At 7 weeks of age, flies expressing LRRK2-1 or LRRK2-G2019S-2 showed significant decreases in anti-TH staining up to 28% and 50%, respectively. In contrast, there were no significant changes in anti-elav and anti-5-HT immunostaining at 5 weeks (FIGS. 5) and 7 weeks (data not shown) of age compared with nontransgenic control flies. These results indicate that expression of LRRK2 proteins was selectively toxic for DA neurons and that LRRK2-G2019S was more toxic than wild-type LRRK2.

### Discussion

Mutations in the LRRK2 gene represent the most common known cause of PD (1, 2, 24). Unlike mutations in other PD-linked genes, LRRK2-linked disease has a clinical progression and neurochemical phenotype similar to that of typical late-onset disease, but little is known about LRRK2-linked molecular pathogenesis. Here we have created gain-of-function LRRK2 Drosophila models by overexpressing the human wild-type LRRK2 and the mutant form LRRK2-G2019S. Expression of both forms of LRRK2 led to retinal degeneration, selective loss of DA neurons in the brain, early mortality, and locomotor impairment. Moreover, LRRK2-G2019S caused a more severe parkinsonism-like phenotype than wild-type LRRK2. Treatment with L-DOPA improved the mutant LRRK2-induced locomotor impairment but did not prevent the loss of TH-positive neurons, similar to LRRK2-linked human PD.

Expression of LRRK2 in all neurons under the control of the elav-GAL4 caused a less severe phonotype in flies than specific expression of LRRK2 in DA neurons under the control of the ddc-GAL4, although the expression levels of proteins in fly head homogenates was higher in elav-GAL4;LRRK2 flies. This paradox may be explained by the lower expression of LRRK2 proteins in DA neurons by elav-GAL4. LRRK2 triggered the loss of anti-TH immunostaining but not the significant loss of anti-elav or anti-5-HT immunostaining, indicating that LRRK2-induced toxicity is preferentially localized to DA neurons in the brain, which is reminiscent of human PD. The manifestation of symptoms in PD patients is associated with the loss of 50-60% of DA neurons (25-27). We found that 5-week-old ddc-GAL4;G2019S-2 flies and 7-week-old elav-GAL4;G2019S-2 flies had an ≈50% reduction of TH-positive neurons. These results indicated that LRRK2 induced the loss of DA neurons or TH expression. In either case there would be loss of DA function. Moreover, the LRRK2 flies displayed parallel kinetics in the loss of TH-positive neurons and locomotor dysfunction, suggesting that these abnormalities may be causally related.

Expression of wild-type LRRK2 protein was toxic, albeit less so than expression of LRRK2-G2019S. These results raise the possibility that an elevated concentration of wild-type LRRK2 protein under some circumstances, such as genetic variation or cellular stress, may lead to DA neuronal degeneration and locomotor impairment and subsequently may contribute to some cases of human PD. As a related example, genetic duplication or triplication at the α-synuclein locus leading to overexpression of wild-type α-synuclein caused PD (28), although there are as yet no similar reports that elevated expression of wild-type LRRK2 links to human PD. A recent report shows that the disease phenotype and mortality of patients with heterozygous versus homozygous G2019S mutations are similar (29). However, there was an expression-level-dependent effect in phenotype between different G2019S fly lines. G2019S-2, which had a higher expression level than G2019S-3, has a faster rate of mortality and locomotor impairment than G2019S-3. At comparable expression levels, the mutant G2019S-LRRK2 had a more severe phenotype than wild-type LRRK2. The G2019S-3 line had much lower expression than wild-type LRRK2-1 but had a faster rate of mortality. We further found that mutant G2019S-2 had higher autophosphorylation activity than wild-type LRRK2-1. These results are consistent with in vitro findings that mutant LRRK2-G2019S has higher protein kinase activity and causes greater toxicity than wild-type LRRK2 (5, 10-14). A recent report shows that the G2019S mutation in LRRK2 appears to increase autophosphorylation through a process that seems to involve reorganization of the kinase activation segment and suggests a molecular explanation for how the G2019S mutation enhances the catalytic activity of LRRK2, thereby leading to pathogenicity (13). Another possibility is that LRRK2 may act as a scaffold protein to alter other signaling molecules leading to pathogenicity through its specific proteinprotein interaction domains (LRR and WD40). Clarifying the effects of PD-associated mutation on kinase activity and PD pathogenesis awaits the identification of true LRRK2 substrates and interaction partners. LRRK2 Drosophila may be a potential useful in vivo system to identify these LRRK2 interactors or substrates.

Drosophila CG5483 (the fly homolog of LRRK2) is expressed in all tissues examined and may be enriched in brain and thoracicoabdominal ganglion according to the FlyBase database (http://flybase.bio.indiana.edu/reports/FBgn0038816.html) by cDNA array. The loss-of-function mutant studies indicate that the Drosophila CG5483 protein is critical for the integrity of fly DA neurons (16). Transgenic expression of Drosophila wild-type CG5483 and a mutation (R1069C) corresponding to the human R1441C mutation does not show any significant defects (16). This mutation in the context of Drosophila CG5483 may not be as pathogenic as the same R1441C change in the context of the human LRRK2 patients. Alternatively, the expression level of this mutant allele may not reach the pathology threshold in the fly. A recent report shows that overexpression of mouse LRRK2 with a mutation corresponding to the R1441C mutation in human LRRK2 results in biochemical features similar to those of human LRRK2-R1441C; whether these mice display motor dysfunction and DA neuronal loss has not been reported (30). Introduction of the human LRRK2 protein kinase domain fragment into adult rat substantia nigra, via adenoassociated virus-2-mediated gene transduction, has been reported to cause of degeneration of DA neurons (5).

To our knowledge, our study is the first report of an animal gain-of-function model expressing full-length human LRRK2. The limitations of our study are that we have used only one mutation (albeit the most common) and that LRRK2 proteins may be overexpressed relative to the endogenous fly LRRK2 homolog. However, flies with lower expression had a phenotype similar to those with higher expression, just less severe. The findings in this study need to be extended to vertebrate animals and compared with human patients. It is noteworthy that the phenotype of the LRRK2 flies recapitulates several key features of the human disorder and can be improved by L-DOPA, thereby representing a valuable genetic model for pathogenesis study of LRRK2-linked parkinsonism. This model may be useful to screen for LRRK2 interactors and to search for LRRK2 substrates. No effective treatments are as yet available to prevent the progressive death of DA neurons in PD. The Drosophila model of mutant α-synuclein has unveiled a gain-of-function mechanism for mutant α-synuclein-linked PD and provided a model for testing neuroprotective strategies for α-synuclein-mediated toxicity (18, 31). Similarly, the LRRK2 flies may provide a useful in vivo model for therapeutic screens to prevent neuronal loss and to rescue locomotor dysfunction in PD.

### REFERENCES

The disclosure of each reference cited is expressly incorporated herein.

1. Paisan-Ruiz, C., Jain, S., Evans, E. W., Gilks, W. P., Simon, J., van der, B. M., Lopez, d. M., Aparicio, S., Gil, A M., Khan, N. et aL. (2004) Neuron 44, 595-600.

2. Zimprich, A., Biskup, S., Leitner, P., Lichtner, P., Farer, M., Lincoln, S., Kachergus, J., Hulihan, M., Uitti, R 1, Caine, D. B. et al. (2004) Neuron 44, 601-607.

3. Ross, O. A., Toft, M., Whttle, A. J., Johnson, J. L., Papapetropoulos, S., Mash, D. C., Litvan, 1., Gordon, M. F., Wszolek, Z. K., Farer, M. J. et al. (2006) Ann. Neural. 59,388-393.

4. Rajput, A, Dickson, D. W., Robinson, C. A, Ross, O. A, Dachsel, J. C., Lincoln, S. J., Cobb, S. A, Rajput, M. L., & Farer, M. L (2006) Neurology 67,1506-1508.

5. MacLeod, D., Dowman, J., Hamond, R, Leete, T., Inoue, K., & Abeliovich, A (2006) Neuron. 52,587-593.

6. Cookson, M. R, Dauer, W., Dawson, T., Fon, E. A, Guo, M., & Shen, J. (2007) J Neurosci 27, 11865-11868.

7. Nichols, W. C., Panatz, N., Hernandez, D., Paisan-Ruiz, C., Jain, S., Halter, C. A, Michaels, V. E., Reed, T., Rudolph, A., Shults, C. W. et al. (2005) Lancet365,410-412.

8. Di Fonzo, A., Rohe, C. F., Ferreira, J., Chien, H. F., Vacca, L., Stocchi, F.,Guedes, L., Fabrizio, E., Manfiedi, M., Vanacore, N. et al. (2005) Lancet 365,412-415.

9. Gilks, W. P., Abou-Sleiman, P. M., Gandhi, S., Jain, S., Singleton, A, Lees, A. J., Shaw, K., Bhatia, K. P., Bonifati, V., Quinn, N. P. et aL. (2005) Lancet 365,415-416.

10. West, A B., Moore, D. J., Biskup, S., Bugayenko, A., Smith, W. W., Ross, C. A, Dawson, V. L., & Dawson, T. M. (2005) Proc. NatL. Acad. Sci. U. S. A. 102,16842-16847.

11. Greggio, E., Jain, S., Kingsbur, A, Bandopadhyay, R, Lewis, P., Kaganovich, A, van der Brug, M. P., Beilina, A, Blackinton, J., Thomas, K. J. et aL. (2006) NeurobioL. Dis. 23,329-341.

12. Smith, W. W., Pei, Z., Jiang, H., Dawson, V. L., Dawson, T. M., & Ross, C. A. (2006) Nat. Neurosci. 9, 1231-1233.

13. Luzon-Toro, B., de la Torre, E. R, Delgado, A., Perez-Tur, 1, & Hilfiker, S. (2007) Hum. MoL. Genet.

14. Jaleel, M., Nichols, R. J., Deak, M., Campbell, D. G., Gilardon, F., Knebel, A., & Alessi, D. R (2007) Biochem. J 405,307-317.

15. Gloeckner, C. J., Kin, N., Schumacher, A, Braun, R J., O'Neil, E., Meitinger, T., Koich, W., Prokisch, H., & Ueffng, M. (2006) Hum. MoL. Genet. 15,223-232.22

16. Cauchi, R. L & van den, H. M. (2006) Neurodegener. Dis. 3, 338-356.

17. Marsh, L L. & Thompson, L. M. (2006) Neuron. 52, 169-178.

18. Feany, M. B. & Bender, W. W. (2000) Nature. 404,394-398.

19. Lee, S. B., Kim, W., Lee, S., & Chung, L (2007) Biochem. Biophys. Res. Commun. 358,534-539.

20. Brand, A H. & Perrmon, N. (1993) Development. 118,401-415.

21. Haass, C. & Kahle, P. L (2000) Nature. 404,341,343.

22. Budnik, V. & White, K. (1988) J. Camp Neural. 268,400-413.

23. Friggi-Grelin, F., Cou10m, H., Meller, M., Gomez, D., Hirsh, J., & Birman, S. (2003) J. NeurobioL. 54,618-627.

24. Hardy, 1, Cai, H., Cookson, M. R., Gwinn-Hardy, K., & Singleton, A. (2006) Ann. Neural. 60,389-398.

25. Dauer, W. & Przedborski, S. (2003) Neuron 39,889-909.

26. Form, L. S. (1996) J. Neuropathol. Exp. Neural. 55,259-272.

27. Mouradian, M. M. (2002) Neurology 58,179-185.

28. Singleton, A. B., Farrer, M., Johnson, J., Singleton, A., Hague, S., Kachergus, J., Hulihan, M., Peuralina, T., Dutra, A, Nussbaum, R. et al. (2003) Science. 302,841.

29. Ishihara, L., Waren, L., Gibson, R., Amouri, R., Lesage, S., Dur, A, Tazir, M., Wszolek, Z. K., Uitti, R.1, Nichols, W. C. et al. (2006) Arch. NeuraL. 63, 1250-1254.

30. Li, X., Tan, Y. C., Poulose, S., Olanow, C. W., Huang, X. Y., & Vue, Z. (2007) J. Neurochem.

31. Auluck, P. K., Meulener, M. C., & Bonin, N. M. (2005) J. BioL. Chem. 280,2873-2878.

32. Li, H., Chaney, S., Roberts, 1. J., Forte, M., & Hirsh, J. (2000) Curro BioL. 10,211-214.

33. Xu, H., Lee, S. J., Suzi, E., Dugan, K. D., Stoddard, A, Li, H. S., Chodosh, L.A, & Montell, C. (2004) EMBOJ. 23, 811-822.

34. Rothenfuh, A., Abodeely, M., Price, J. L., & Young, M. W. (2000) Genetics. 156,665-675.

35. Chaudhuri, A, Bowling, K., Funderburk, C., Lawal, H., Inamdar, A, Wang, Z., & O'Donnell, L M. (2007) J. Neurosci 27,2457-2467.

36. Monastirioti, M. (1999) Microsc. Res. Tech. 45, 106-121.

We claim:

1. A transgenic fly whose genome comprises a human wild-type leucine-rich repeat kinase 2 (LRRK2) gene, wherein expression of said gene creates a parkinsonism-like phenotype and wherein said fly exhibits selective dopaminergic (DA) neuron loss, retinal degeneration, locomotor dysfunction and premature mortality.

2. A transgenic fly whose genome comprises a mutant human LRRK2 gene, wherein expression of said gene creates a parkinsonism-like phenotype and said fly exhibits selective dopaminergic (DA) neuron loss, retinal degeneration, severe locomotor dysfunction and premature mortality.

3. The transgenic fly of claim 2, wherein the mutant human LRRK2 gene is a G2019S mutation.

4. The transgenic fly of claim 2, wherein the mutant human LRRK2 gene is an 12020T mutation.

5. A method of screening compounds for the ability to modulate activity of a LRRK2 protein expressed in the transgenic flies of claim 1 or 2, and reduce changes associated with the parkinsonism-like phenotype induced by LRRK2 transgene expression, comprising the steps of:
(a) exposing the transgenic fly to an effective amount of a candidate compound to modulate activity of the LRRK2 protein, and
(b) determining whether there is significant effect of said compound on the parkinsonism-like phenotype of the fly as compared to a fly of claim 1 or 3 that was not exposed to said compound,
wherein a compound that has an effect on the parkinsonism-like phenotype of the fly induced by activity of the expressed LRRK2 protein is identified as a candidate compound for modulating activity of a LRRK2 protein.

6. A method of screening compounds for the ability to modulate activity of a LRRK2 protein expressed in the transgenic flies of claim 1 or 2, and reduce changes associated with the parkinsonism-like phenotype induced by LRRK2 transgene expression, comprising the steps of:
(a) exposing the transgenic fly to an environmental stressor to accelerate expression of the parkinsonism-like phenotype,
(b) exposing the transgenic fly to an effective amount of a candidate compound to modulate activity of the LRRK2 protein, and
(c) determining whether there is a significant effect of said compound on the parkinsonism-like phenotype of the fly as compared to a fly of claim 1 or 3 that was not exposed to said compound,
wherein a compound that has an effect on the parkinsonism-like phenotype of the fly induced by activity of the expressed LRRK2 protein is identified as a candidate compound for modulate activity of a LRRK2 protein.

7. The method of claim 6, wherein the environmental stressor comprises the members of the group consisting of: a temperature in a range of 25° C.-29 ° C., H₂O₂, intracellular stressors and extracellular stressors.


SEQUENCE LISTING (4 sequences)

SEQ ID NO: 1 - DNA, 24 bp, Artificial Sequence
  Description of Artificial Sequence Synthetic primer
cggcctatttaaacgccacagcaa

SEQ ID NO: 2 - DNA, 24 bp, Artificial Sequence
  Description of Artificial Sequence Synthetic primer
aactgaagtgttgcgcgaagaacc

SEQ ID NO: 3 - DNA, 24 bp, Artificial Sequence
  Description of Artificial Sequence Synthetic primer
attgcgaacctggatgtctctcgt

SEQ ID NO: 4 - DNA, 24 bp, Artificial Sequence
  Description of Artificial Sequence Synthetic primer
tcaggcacgaagctcagctgatta